(19) United States
(12) Patent Application Publication
(10) Pub. No.: US 2018/0340214 A1
(43) Pub. Date: Nov. 29, 2018

(54) QUANTITATIVE MULTIPLEX POLYMERASE CHAIN REACTION IN TWO REACTIONS

(71) Applicant: DIATHERIX Laboratories, Inc., Huntsville, AL (US)

(72) Inventors: Stefan BRZEZINSKI, Decatur, AL (US); Danielle JOHNSON, Huntsville, AL (US); Cheryl SESLER, Athens, AL (US); Elena GRIGORENKO, Madison, AL (US)

(73) Assignee: DIATHERIX Laboratories, Inc., Huntsville, AL (US)

(21) Appl. No.: 15/605,401

(22) Filed: May 25, 2017

(51) Int. Cl. C12Q 1/68

(52) U.S. Cl. CPC C12Q 1/686; C12Q 1/6851

(57) ABSTRACT

Methods are disclosed herein for quantitative multiplex PCR. The methods proceed through at least two successive rounds of amplification, for example, a target enrichment step and a target amplification step.

### FIELD OF THE INVENTION

The invention described herein relates to multiplex polymerase chain reaction (PCR) methods, including multiplex methods that can quantify the concentration of individual nucleotide templates in a sample.

### BACKGROUND

Multiplex PCR allows the amplification of multiple loci, potentially from multiple organisms, in one reaction using multiple sets of locus specific primers. However, multiplex PCR has historically been limited by cross reactivity and mutual inhibition among the various primer pairs. U.S. Pat. No. 7,851,148 to Han discloses compositions and methods that can overcome these problems to provide a more reliable and robust multiplex PCR system. Han designates this PCR technology target-enriched multiplex (“TEM”) PCR. However, one limit of TEM PCR is that it only provides information about the presence or absence of a target sequence in a reaction mix at the end of the amplification. That is to say, TEM PCR as described by Han is an “end-point” PCR technology.

So-called “real-time” PCR technologies are useful for quantifying target nucleotides. For this reason, real-time PCR is also frequently designated “quantitative PCR” or “qPCR.” See, e.g., U.S. Pat. No. 7,972,828 to Ward et al. However, for qPCR to be run in multiplex fashion has historically required even more careful balancing than end-point multiplex PCR. See, e.g., U.S. Pat. No. 5,876,978 to Willey et al. In addition quantitative multiplex PCR is unable to use the more convenient features of non-multiplex qPCR, such as fluorescence intensity read-outs from SYBR™ dyes.

### SUMMARY

The present disclosure describes compositions and methods that are useful for multiplex qPCR. In certain embodiments, the PCR reactions described herein can be monitored in real-time based on gradually increasing fluorescence intensity. In alternative embodiments, the PCR reactions provide data only at the end of the reaction process. Further, in certain embodiments the process requires two chemically distinct reaction mixtures to run to completion.

For example, in one embodiment a method for quantitative multiplex amplification is disclosed herein, in which the method comprises: (a) amplifying a plurality of target sequences in a first reaction mix, and (b) amplifying the plurality of target sequences in a cycler containing a second reaction mix. In this embodiment, the first reaction mix comprises, for each target sequence in the plurality, (i) a first pair of target enrichment primers comprising a forward outer (Fₒ) and a reverse outer (Rₒ) primer that each hybridize to a sequence adjacent to the target sequence; and (ii) a second pair of target enrichment primers comprising a forward inner (Fᵢ) and a reverse inner (Rᵢ) primer, each of which hybridize to a portion of the target sequence. The Fᵢ primers comprise a tag comprising a sequence complementary to a portion of one of a pair of target amplification primers and the Rᵢ primers comprise a tag comprising a sequence complementary to a portion of the other of the pair of target amplification primers. In this embodiment, the step a) amplification generates a plurality of first amplification products. The second reaction mix comprises: (i) the target amplification primers, in which the pair of target amplification primers comprises a forward super primer (FSP) and a reverse super primer (RSP), each of which binds to its corresponding tag on the first amplification products; (ii) a dilution of the first amplification products from step a), diluted at least two fold relative to the concentration of first amplification products at the end of step a); (iii) a thermostable DNA polymerase with 5′ to 3′ exonuclease activity; and (iv) a plurality of sequence-specific probes, wherein there is at least one probe complementary to each target sequence in the plurality, and wherein each sequence-specific probe comprises at least one fluorophore and a quencher. The at least one fluorophore responds to an excitation wavelength by emitting a first fluorescence and the quencher quenches the first fluorescence prior to hydrolysis of the probe. The cycler is equipped with filters responsive to a plurality of first fluorescence wavelengths. An illumination source periodically illuminates the second reaction mix for detection.

In another embodiment a method for quantitative multiplex amplification is disclosed herein, in which the method comprises: (a) amplifying a plurality of target sequences in a first reaction mix, and (b) amplifying the plurality of target sequences in a cycler containing a second reaction mix. For each target sequence in the plurality, the first reaction mix comprises: (i) a first pair of target enrichment primers comprising a Fₒ and a Rₒ primer that each hybridize to a sequence adjacent to the target sequence; and (ii) a second pair of target enrichment primers comprising a Fᵢ and a Rᵢ primer, each of which hybridize to a portion of the target sequence. The Fᵢ primers comprise a tag comprising a sequence complementary to a portion of one of a pair of target amplification primers and the Rᵢ primers comprise a tag comprising a sequence complementary to a portion of the other of the pair of target amplification primers. The step (a) amplification generates a plurality of first amplification products. The second reaction mix comprises: (i) the target amplification primers, in which the pair of target amplification primers comprises a forward super primer (FSP) and a reverse super primer (RSP), each of which binds to its corresponding tag on the first amplification products; (ii) a dilution of the first amplification products from step a), diluted at least two fold relative to the concentration of first amplification products at the end of step a); and (iii) a fluorescent dye that emits a more intense fluorescence at a given wavelength when bound to double-stranded DNA (dsDNA) than when not bound to dsDNA. The cycler is equipped with filters responsive to a plurality of first fluorescence wavelengths. An illumination source periodically illuminates the second reaction mix for detection.

Additional details and exemplary embodiments are disclosed hereinafter.

### BRIEF DESCRIPTION OF THE DRAWINGS

FIG. 1 shows a flow chart of a multiplex PCR method.

FIG. 2 shows a flow chart of a multiplex PCR method. Although SYBR Green® is shown in FIG. 2 for purpose of illustrative example, the method described is not limited to embodiments with SYBR Green®.

FIG. 3 illustrates data from real-time PCR tests. Panel A shows the results when probe directed against wild-type 23S DNA is used to detect wild-type (38770 & 38771) template and not mutant (38772 & A2142G) template as amplification curves are below the threshold. Panel B shows the results when probe directed against A2143G mutant 23S DNA is used to detect A2143G mutant (38772) template and not A2142G mutant (A2142G) or wild-type (38770 & 38771) template as amplification curves cross the threshold at later cycles. Panel C shows detection of A2142G mutant template with A2142G probe. Panel D shows detection of all H. pylori templates with the ureA probe.

FIG. 4 shows results from amplifications of wild-type H. pylori template using 23S wild-type specific primers and different concentrations of probes specific to wild-type 23S.

### DETAILED DESCRIPTION

As used herein, all nouns in singular form are intended to convey the plural and all nouns in plural form are intended to convey the singular, except where context clearly indicates otherwise. As used herein, “and/or” includes any and all combinations of one or more of the associated listed items.

In certain embodiments, the methods and compositions described herein may be used to diagnose disease agents. As used herein, an “agent” means any organism, regardless of form, that incorporates a nucleic acid and that causes or contributes to an infection, a symptom, or a condition, including, but not limited to a bacteria, a virus (regardless of RNA or DNA genome), a fungus, or a eukaryotic parasite. The infection, symptom, or condition is designated a “disease state” herein. As used herein, a “disease agent” is an agent that causes or contributes to a disease state. In certain embodiments the disease agent may be involved in bio-weapons programs, such as the organism described as potential biothreats in the 2007 NIAID Biodefense Research Agenda Update or the NIAID's 2014 Public Health Emergency Medical Countermeasures Enterprise (PHEMCE) Strategy and Implementation Plan.

The methods and compositions described herein are useful in detecting and/or quantifying one or more “target” sequences. As used herein, a “target” sequence is any sequence whose presence, absence, or concentration in a sample can provide useful information for making a given determination (e.g., “does this patient have a Staphylococcus aureus infection?”, or “is this pool water contaminated with Giardia lamblia?”). In certain embodiments, the methods described herein can be used to detect “at least one target.” In the context of the present specification “at least one target” implies at least two sequences to be detected, viz. the target of interest that conveys information about the study sample (e.g., a patient sample or an environmental sample) and a control sequence whose measurement makes it possible to interpret the results regarding the target sequence(s) of interest.

As used interchangeably in this disclosure, “nucleic acid molecule,” “oligonucleotide,” and “polynucleotide” include RNA or DNA, whether single or double stranded, and regardless of whether coding, complementary, or antisense. “Nucleic acid molecule,” “oligonucleotide,” and “polynucleotide” also include RNA/DNA hybrid sequences in either single chain or duplex form. As used herein, “nucleotide” can be an adjective to describe molecules comprising RNA, DNA, or RNA/DNA hybrid sequences of any length. More precisely, “nucleotide sequence” encompasses the nucleic material itself and is thus not restricted to the sequence information (i.e., the succession of nucleotide bases) that biochemically characterizes a specific DNA or RNA molecule. As used herein, “nucleotide” can also be a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. As used herein, “modified nucleotides” comprise at least one modification such as (a) an alternative linking group, (b) an analogous form of purine, (c) an analogous form of pyrimidine, or (d) an analogous sugar. Polynucleotide sequences described herein may be prepared by any known method, including synthetic, recombinant, ex vivo generation, or a combination thereof, as well as any purification methods known in the art.

The compositions and methods described herein are capable of detecting disease agents that cause or contribute to a variety of disease states. In particular, these compositions and methods can be used in differential diagnosis to determine if a specific disease agent is present and to determine if secondary disease agents are present. However, the compositions and method described herein may also be used to determine the presence or absence of genetic mutations related to disease states, the presence or absence of single nucleotide polymorphisms (SNPs), to determine gene expression profiling, and to determine gene dosage mutations. These compositions and methods can also be used to determine the presence, absence, or concentration of a biologic agent in a given environmental setting, such as a drinking water reservoir, a pond, a lake, a beach, a sewage treatment plant, a feed lot, a food supply, and/or a beverage supply. Applications of these alternative uses are described in US 2004/0086867. Other uses of the compositions and methods described herein will be apparent to those skilled in the art.

All patents and published patent applications referenced herein are incorporated by reference in their entireties. Where definitions conflict as between the present text and texts incorporated by reference, the definitions of the present text control.

### Nucleic Acid Isolation

In certain embodiments, nucleic acids can be isolated prior to detection. In various embodiments, both RNA and DNA are isolated in a single reaction. In other embodiments, RNA and DNA may be isolated independently. In additional embodiments, the individually isolated DNA and RNA can be combined prior to detection. A variety of techniques and protocols are known in the art for RNA and/or DNA isolation. The nucleic acid isolation techniques may be used to isolate nucleic acid from a variety of patient samples or sources. Such patient samples/sources include, but are not limited to, nasal/pharyngeal swabs, saliva, sputum, serum, whole blood and stool.

In certain embodiments, nucleic acid isolation may inactivate infectious agents in the sample, thus reducing any risk to laboratory and healthcare personnel. In such circumstances, requirements for stringent bio-containment procedures may also be relaxed for the remaining steps of the PCR analysis. In addition, DNA and/or RNA isolation may remove enzymatic inhibitors and other unwanted compounds from the isolated nucleic acid, thus making the subsequent PCR more efficient.

In one embodiment, a dual RNA/DNA isolation method is used employing an affinity resin (e.g., QIAGEN® DNEasy® and/or RNEasy® technologies) for initial isolation of RNA and/or DNA from patient samples. Wash steps may be used to remove PCR and RT-PCR inhibitors. The column method for nucleic acid purification is advantageous as it can be used with different types of patient samples and the spin and wash steps effectively remove PCR or RT-PCR inhibitors.

### Reverse Transcription

Additionally or alternatively, where an RNA genome or an RNA target is present, reverse transcription (“RT”) PCR may be utilized. PCR and RT-PCR methodologies are well known in the art.

### Probe-Based Detection

In certain embodiments, amplification products may be detected using fluorescently labeled nucleotide probes. The probes can contain sequences complementary to the amplicon to be detected. In certain embodiments, the probes may also contain non-complementary sequences at one or both ends. The complementary sequence of each probe can be any length (e.g., at least 5 bp, at least 10 bp, at least 15 bp, at least 20 bp, at least 25 bp, at least 30 bp, at least 35 bp, at least 40 bp, at least 45 bp, or at least 50 bp), but the person of ordinary skill will appreciate that the length of the complementary sequence will affect both melting temperature and target specificity. The probes may contain a molecule capable of fluorescence at a given excitation wavelength (“fluorophore”) on one end and a molecule capable of quenching the fluorophore's fluorescence (a “quencher”). For example, a given probe might contain OREGON GREEN® on one end and BHQ1® on the other. Additionally or alternatively, a given probe might contain CY3® on one end and BHQ2® on the other. Additionally or alternatively, a given probe might contain CY5® on one end and BHQ3® on the other. Additionally or alternatively, a given probe might contain FAM® on one end and QSY® on the other. Additionally or alternatively, a given probe might contain VIC® on one end and QSY® on the other. Additionally or alternatively, a given probe might contain FAM® on one end and MGB® on the other. Additionally or alternatively, a given probe might contain NED® on one end and MGB® on the other. Additionally or alternatively, a given probe might contain JUN® on one end and QSY® on the other. Additionally or alternatively, a given probe might contain CY5® on one end and QSY® on the other. Different quenchers require different degrees of physical proximity to their respective fluorophores, and the person of ordinary skill will know how to adjust the length of the probe as necessary to ensure that the quencher is properly positioned relative to the fluorophore.

### PCR Procedures

a. Target Enrichment Step

The methods described herein allow for efficient amplification of multiple target sequences without extensive empirical testing of primer combinations and amplification conditions as is required with other multiplex amplification methods known in the art. Detection of an amplified target sequence indicates the presence and identity of the target in the sample of interest, thereby providing useful information. In one embodiment, a single target sequence is selected for amplification from each of a set of disease agents to be tested. In an alternate embodiment, more than one target sequence is selected for amplification from each disease agent in the set.

In the methods described herein, the process occurs in two steps. In the first, various target sequences are amplified in a target enrichment step. After the target enrichment, the amplification products can be diluted before the second, target amplification step. In certain embodiments, probes, primers, and/or enzymes can be added after the dilution but before the start of the target amplification step.

For target enrichment, the user provides a series of target enrichment primers for each of a set of target sequences to be analyzed. Each target is defined by at least four “target enrichment” primers: a forward outer primer (Fₒ); a reverse outer primer (Rₒ); a forward inner primer (Fᵢ); and a reverse inner primer (Rᵢ). The Fᵢ primer is substantially identical in sequence to the 5′ end of the top strand of the target sequence, while the Rᵢ primer is substantially reverse complementary to the 3′ end of the top strand of the target sequence. As a result, the Fᵢ and Rᵢ primers can each hybridize to a portion of the target sequence. The Fᵢ and Rᵢ primers can each be any length greater than 5 nucleotides, for example greater than 10, greater than 15, greater than 20, greater than 25, greater than 30, greater than 35, greater than 40, greater than 45, or even greater than 50 nucleotides in length. In practice, Fᵢ and Rᵢ primers will each typically be no less than 12 nucleotides in length but no more than 45 nucleotides in length. The Fᵢ and Rᵢ primers do not have to be each identical in length to the other, but it is useful that the melting temperatures for each be no more than 5 C° apart, for example no more than 3 C°, no more than 2 C°, or no more than 1 C° apart. In certain embodiments, the Fᵢ and Rᵢ primers will have identical melting temperatures. Those of ordinary skill know how to calculate the melting temperature of a PCR primer, and will thus understand that the melting temperature is proportional both to the total length of the primer and to the G/C content.

Although various embodiments of the methods and compositions disclosed herein involve multiple Fᵢ/Rᵢ primer sets, in which each Fᵢ/Rᵢ primer set binds to a unique target, various Fᵢ/Rᵢ primer sets share a common set of tags at the 5′ end of each primer. In certain embodiments, all Fᵢ/Rᵢ primer sets share a common set of tag sequences.

The Fₒ primer is substantially identical in sequence to a sequence adjacent to but upstream of the top strand of the target, while the Rₒ primer is substantially reverse complementary to a sequence adjacent to but downstream of the top strand of the target. As used herein, “adjacent” is not limited to “immediately adjacent.” For example, in the sequence A-B-C-D-E-F-G . . . [etc], A is immediately adjacent to B, but not adjacent to G. However, in certain circumstances, it is appropriate to describe A as being adjacent to C, even though A is not immediately adjacent to C. With more specific reference to nucleotides, to say that the Fₒ primer and/or the Rₒ primer is “adjacent” to the target means that the primers each bind to a sequence near enough to the target that the primers can still prime amplification of the target, even though the location of primer binding may not be immediately adjacent to the target. The Fₒ and Rₒ primers do not have to be each identical in length to the other, but it is useful that the melting temperatures for each be no more than 5 C° apart, for example no more than 3 C°, no more than 2 C°, or no more than 1 C° apart. In certain embodiments, the Fₒ and Rₒ primers will have identical melting temperatures.

The specificity of annealing between the target enrichment primers and their nucleic acid sequences can be adjusted by increasing or decreasing the length of the primer sequence responsible for annealing as is known in the art. In general, a longer primer sequence will give increased specificity. Increasing or decreasing the lengths of the primer sequence responsible for annealing may also determine which primers are active during the various stages of the amplification process. In various embodiments, the length of the target enrichment primers can be from 10 to 50 nucleotides, from 10 to 40 nucleotides, or from 10 to 20 nucleotides. Each target enrichment primer may be a different length from the others if desired. For example, in one embodiment, the Fₒ and Rₒ are each 15-25 nucleotides in length, while Fᵢ and Rᵢ are each 35-45 nucleotides in length.

The target enrichment primers can be used at low concentrations for enrichment (i.e. limited amplification) of the target sequence. The concentration of the target enrichment primers need not be sufficient for exponential amplification of the target sequences. As used herein, a “low concentration” when used to describe the concentration of the target enrichment primers means a concentration of primers that is not sufficient for exponential amplification of the given target sequence(s), but which is sufficient for target enrichment of the given target sequences. This low concentration may vary depending on the nucleotide sequence of the nucleic acid containing the target sequence to be amplified. In one embodiment, a concentration of target enrichment primers is in the range of 2 nM to less than 200 nM. In another embodiment, a concentration of target enrichment primers is in the range of 2 nM to 150 nM. In an alternate embodiment, a concentration of target enrichment primers is in the range of 2 nM to 100 nM. In yet another alternate embodiment, a concentration of target enrichment primers is in the range of 2 nM to 50 nM. Other concentration ranges outside those described above may be used if the nature of the nucleic acid sequence containing the target sequence to be amplified is such that concentrations of target enrichment primers below or above the ranges specified are required for target enrichment without exponential amplification. The various target enrichment primers may be used in different concentrations (i.e. ratios of forward to reverse primer) or at the same concentration.

Multiple sets of target enrichment primers may enhance the sensitivity and specificity of the assay by allowing more opportunity and combinations for the nested primers to work together to provide target sequence enrichment. For example, more than two sets of target enrichment primers may be used if desired. In various embodiments, 3 to 6 sets of target enrichment primers can be used, or 3 to 5 sets of target enrichment primers can be used, or 3 to 4 sets of target enrichment primers can used.

Following the target enrichment step, the reaction mix containing a plurality of first amplification products—such as the target enrichment amplification products—can be diluted. Without being bound by theory, this dilution can be useful to reduce inhibitors from the target enrichment step that might otherwise interfere with the target amplification step. Dilution can be done to any degree greater than or equal to at least about half-again, for example at least about 2-fold, at least about 5-fold, at least about 7.5 fold, at least about 10-fold, at least about 15-fold, or at least about 20-fold.

b. Target Amplification Step (Exponential Amplification Step)

The next step of the processes disclosed herein involves amplifying a plurality of target sequences in a cycler. The cycler contains a second reaction mix. Target amplification employs at least one set of “target amplification” primers designated “forward super primer” (FSP) and “reverse super primer” (RSP). The target amplification primers are not present in the first reaction mix during the target enrichment step, but rather are added to the second reaction mix at the start of the target amplification step. In certain embodiments, polymerase is added to the second reaction mix before the start of the target amplification step, for example shortly after the dilution, and/or at substantially the same time as the target amplification primers are added. In some embodiments, the second reaction mix polymerase can be a polymerase with an exonuclease activity, such as Taq polymerase. In certain embodiments, probes as described herein above can be added to the reaction mix before the start of the target amplification step, for example shortly after the dilution, and/or at substantially the same time as the fresh polymerase and/or the target amplification primers is/are added. The exponential amplification of the target sequences is accomplished using the FSP and RSP.

In some embodiments, more than one set of target amplification primers may be used. When more than one set of target amplification primers are used, the sequences of the multiple sets of target amplification primers are selected so that they are compatible with one another in the exponential amplification step. In other words, the multiple sets of super primers would share similar melting temperatures when binding to the super primer binding sites on the amplified target nucleic acid and have similar amplification efficiencies. Multiple target amplification primers may be used when one or more of the detection targets are present at different titers/concentrations. If there is a significant difference in titer, then with only one set of target amplification primers, preferential amplification of the high titer agent may occur. This could result in a false negative diagnosis for the agent present at the lower titer. Such biased amplification may be avoided by using multiple sets of target amplification primers. In various embodiments, 2-4 sets of target amplification primers are used.

The sequence of the target amplification primers are the same for each target sequence to be amplified if one set of target amplification primers are used, or the target amplification primers are designed to share similar amplification characteristics for each target sequence to be amplified if multiple sets of target amplification primers are used. In one embodiment, both of the target amplification primers incorporate a means for detection (e.g., a biotin tag, an enzyme label, a fluorescent tag, a radionucleotide label, etc.) that enables the amplified products to be detected and/or manipulated as described below. In an alternate embodiment, only 1 of the two target amplification primers incorporates a means for detection. In yet another alternate embodiment, only the RSP of the target amplification primers incorporates a means for detection. In one embodiment, the means for detection may be a fluorescent element, such as, but not limited to, a CY-3® label. The fluorescent element may be directly conjugated to the super primer sequences or may be indirectly conjugated (e.g., a biotinylated primer and streptavidin-conjugated fluorophore).

The target amplification primers can be used at high concentration for exponential amplification of the target sequences. The FSP can bind the tag sequence on the Fᵢ primer and the RSP binds the tag sequence on the Rᵢ primer. In other words, FSP/RSP recognize common primer sequences, and thus can amplify all nucleic acids that had been amplified during the target enrichment step. In various embodiments, the target amplification primers are each from 10 to 50 nucleotides in length, or from 10 to 40 nucleotides in length, or from 10 to 20 nucleotides in length.

As used herein, a “high concentration” when used to described the concentration of the target amplification primers (FSP and RSP) means a concentration of primers that is sufficient for exponential amplification of the given target sequence. In one embodiment, a concentration of target amplification is in the range of 200 nM to 2.0 μM. In another embodiment, a concentration of target amplification primers is in the range of 200 nM to 1.0 μM. In an alternate embodiment, a concentration of target amplification primers is in the range of 200 nM to 800 nM. In yet another alternate embodiment, a concentration of target amplification primers is in the range of 200 nM to 400 nM. Other concentration ranges outside those described above may be used if the nature of the nucleic acid sequence containing the target sequence to be amplified is such that concentrations of target amplification primers below or above the ranges specified are required for exponential amplification. The super primers may be used in different concentrations (i.e. ratios of forward to reverse primer) or at the same concentration.

As a general rule, target enrichment primers are at approximately 20 nM, super primers at approximately 750 nM, and probes at approximately 250 nM. A primer concentration in the range of 7500 nM is generally used as a starting point to achieve exponential amplification of a given target sequence. The target enrichment primers and the target amplification primers may be used in various ratios to each one another as discussed herein.

The ratios of the target enrichment primers (Fₒ, Fᵢ, Rₒ, and Rᵢ) used in the amplification method may be varied. Different target sequences may have different target enrichment primer requirements. Some disease agents may have DNA genomes or RNA genomes (positive or negative strand). In addition, the concentration of target amplification primers may also be varied, especially if only one of the super primers is conjugated to a means for detection.

Because target amplification primers are used for the exponential amplification of each target sequence, target amplification primer sequences can be selected so as not to share obvious homology with any known GenBank sequences. In addition, the sequence of the target amplification primers can be selected so as to share a comparable Tₘ on binding to the super primer binding sites in the amplification products to provide efficient amplification reactions. Finally, the sequence of the target amplification primers may be selected such that their priming capabilities for thermal stable DNA polymerases maybe superior to the target enrichment primers which are specific for each target sequence to be amplified.

d. Target Sequences

In certain embodiments, the target sequence will be drawn from an influenza virus, for example a strain selected from the group consisting of H1N1, H1N2, H2N2, H3N2, H3N8, H4N6, H4N8, H5N1, H5N2, H5N3, H6N1, H6N2, H6N4, H7N1, H7N2, H7N3, H7N7, H7N8, H9N2, H10N5, H11N1, H11N8, and H11N9 (which includes the currently circulating avian influenza A strain, H5N1). Additionally or alternatively, the target sequence will be drawn from an adenovirus. Additionally or alternatively, the target sequence will be drawn from members of the Picornaviridae family, which includes enteroviruses and rhinoviruses. Enteroviruses also include different genera such as coxsackie viruses and echoviruses. Additionally or alternatively, the target sequence will be drawn from a bacterium, for example a Helicobacter species, Neisseria meningitides, Haemophilus influenzae, Escherichia coli, Listeria monocytogenes, Mycoplasma pneumoniae, Streptococcus pneumoniae, and Streptococcus agalactiae. Additionally or alternatively, the target sequence will be drawn from a virus selected from the group consisting of enteroviruses, coxsackievirus A, coxsackievirus B, parechovirus, and West Nile virus. Additionally or alternatively, the target sequence will be drawn from a genetic determinant of antibiotic resistance, such as clarithromycin resistance.

e. Kit

A subject of the present disclosure is also a kit comprising the components necessary for carrying out the method disclosed in all the embodiments illustrated. The kit may comprise one or more of the following: at least one set of primers to for the amplification of target sequences from a disease agent and secondary disease agent in sample from an individual suspecting of harboring the disease agent, reagents for the isolation of nucleic acid (RNA, DNA or both), reagents for the amplification of target nucleic acid from said sample (by PCR, RT-PCR or other techniques known in the art), microspheres, either with or without conjugated capturing reagents (in one embodiment, the cRTs), target sequence specific detection oligonucleotides, reagents required for positive/negative controls and the generation of first and second signals.

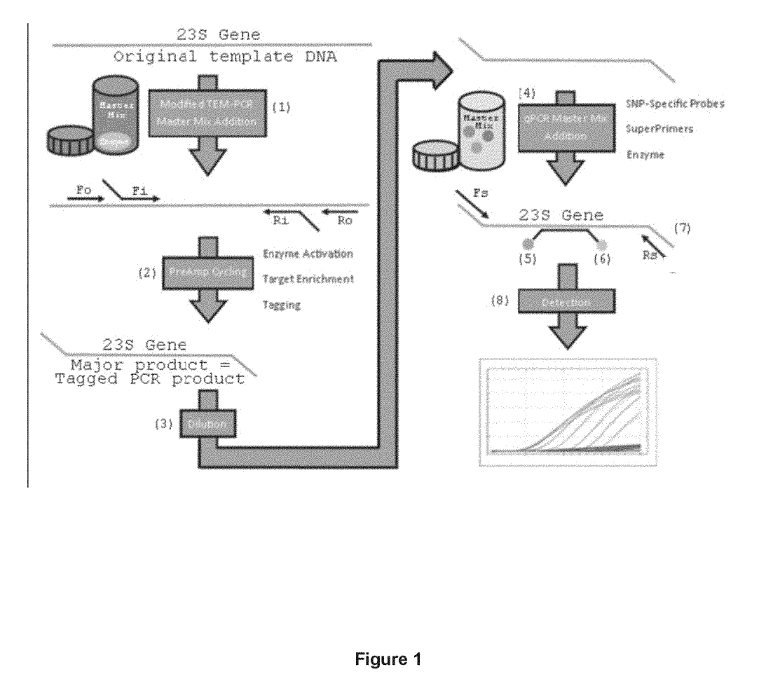

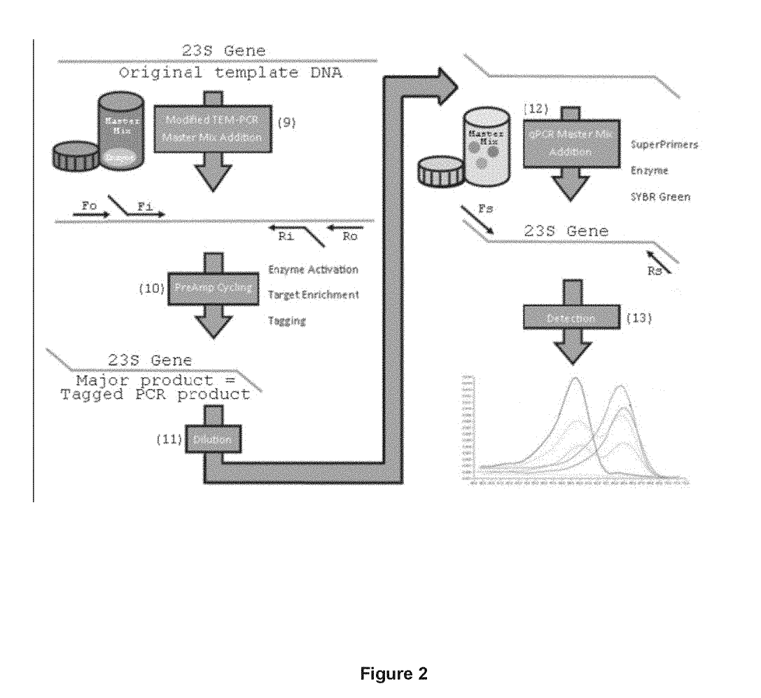

f. FIGS. 1 and 2

FIG. 1 illustrates an exemplary PCR method as described herein. The method begins with a target enrichment step: (1) with the addition of a series of target enrichment primers to a solution containing a template, along with DNA polymerase, deoxyribonucleic acid tri-phosphates (dNTPs), and various salts as necessary to create conditions appropriate for a given PCR amplification. The resulting mixture is cycled (2) through a series of temperatures in a sufficient number of cycles to achieve a set of pre-amplification products, each of which is tagged with the necessary tag sequences from the Fᵢ and Rᵢ primers. At the end of this (2) target enrichment process, the resulting mixture is diluted (3) to create the template mixture for the amplification step. A new set of primers—the target amplification primers—are then added (4), along with the DNA polymerase, dNTPs, and appropriate salts. In addition, probes complementary to the target sequence (e.g., single nucleotide polymorphism (SNP)-specific probes) are added (4) at this stage. Each probe is complementary to its respective tagged amplification product. Each probe carries a fluorescent molecule (5) at one end and a quencher (6) at the other end that quenches the fluorescence from the fluorophore. The DNA polymerase can be a polymerase, such as Taq polymerase, with an exonuclease activity. As the polymerase moves from the target amplification primers along the templates (7), the polymerase will degrade the probe, which is bound to the template and occludes the polymerase's progress along the template. Degradation of the probe, in turn, releases the quencher and/or fluorophore from the probe, such that the fluorophore's fluorescence is no longer quenched, and can be detected (8) by sensors on the lid of the amplification chamber. Thus the amplification of each template can be monitored in real time (8) by following the fluorescence intensity of the corresponding probe. Because each probe can be labeled with its own color, the number of species monitored is limited only by the number of sensors available on the amplification chamber's lid. For example, in some embodiments the chamber lid can monitor at least two different fluorescent colors, for example at least 5, at least 10, at least 15, at least 20, at least 25, or at least 30 different fluorescent colors.

FIG. 2 illustrates another exemplary PCR method as described herein. The method begins with a target enrichment step: (9) with the addition of a series of target enrichment primers to a solution containing a template, along with DNA polymerase, deoxyribonucleic acid tri-phosphates (dNTPs), and various salts as necessary to create conditions appropriate for a given PCR amplification. The resulting mixture is cycled (10) through a series of temperatures in a sufficient number of cycles to achieve a set of pre-amplification products, each of which is tagged with the necessary tag sequences from the Fᵢ and Rᵢ primers. At the end of this (10) target enrichment process, the resulting mixture is diluted (11) to create the template mixture for the target amplification step. A new set of primers—the target amplification primers—are then added (12), along with the DNA polymerase, dNTPs, and appropriate salts, but no probes. Instead of probes, a fluorescent intercalating dye, such as SYBR Green® is added. By way of non-limiting illustration, in certain embodiments the fluorescent dye can be N′,N′-dimethyl-N-[4-[(E)-(3-methyl-1,3-benzothiazol-2-ylidene)methyl]-1-phenylquinolin-1-ium-2-yl]-N-propylpropane-1,3-diamine, AOAO-12, ATTO-633, ATTO-647N, or ATTO-655. Because the intercalating dye binds to all dsDNA, the embodiment illustrated in FIG. 2 cannot be used to track different target species in real time. However, at the end of the target amplification process, the relative prevalence of each target amplicon can be assessed by a size and sequence-based assay (13), such as a melting curve. In this way, the quantitative presence of individual targets in the multiplex template mix can be assessed at the end of the target amplification process.

### EMBODIMENTS

### Embodiment 1

A method for quantitative multiplex amplification, the method comprising:
a. amplifying a plurality of target sequences in a first reaction mix, wherein for each target sequence in the plurality, the first reaction mix comprises:
   i. a first pair of target enrichment primers comprising a forward outer (Fₒ) and a reverse outer (Rₒ) primer that each hybridize to a sequence adjacent to the target sequence; and
   ii. a second pair of target enrichment primers comprising a forward inner (Fᵢ) and a reverse inner (Rᵢ) primer, each of which hybridize to a portion of the target sequence, wherein Fᵢ primers comprise a tag comprising a sequence complementary to a portion of one of a pair of target amplification primers and wherein Rᵢ primers comprise a tag comprising a sequence complementary to a portion of the other of the pair of target amplification primers; and
wherein the step a) amplification generates a plurality of first amplification products; and
b. amplifying the plurality of target sequences in a cycler containing a second reaction mix, wherein the second reaction mix comprises:
   i. the target amplification primers, in which the pair of target amplification primers comprises a forward super primer (FSP) and a reverse super primer (RSP), each of which binds to its corresponding tag on the first amplification products;
   ii. a dilution of the first amplification products from step a), diluted at least two fold relative to the concentration of first amplification products at the end of step a);
   iii. a thermostable DNA polymerase with 5′ to 3′ exonuclease activity; and
   iv. a plurality of sequence-specific probes, wherein there is at least one probe complementary to each target sequence in the plurality, and wherein each sequence-specific probe comprises at least one fluorophore and a quencher, wherein the at least one fluorophore responds to an excitation wavelength by emitting a first fluorescence, and wherein the quencher quenches the first fluorescence prior to hydrolysis of the probe,
wherein the cycler is equipped with filters responsive to a plurality of first fluorescence wavelengths, and wherein an illumination source periodically illuminates the second reaction mix for detection.

### Embodiment 2

The method of embodiment 1, wherein FSP binds the complement of the tag on Fᵢ and RSP binds the complement of the tag on Rᵢ.

### Embodiment 3

The method of any one of the previous embodiments, wherein the length of each of the first pair of target enrichment primers is about 10 to about 100 nucleotides.

### Embodiment 4

The method of any one of the previous embodiments, wherein the length of each of the second pair of target enrichment primers is about 10 to about 100 nucleotides.

### Embodiment 5

The method of any one of the previous embodiments, wherein the length of each of the target amplification primers is about 10 to about 100 nucleotides.

### Embodiment 6

The method of any one of the previous embodiments, wherein the target enrichment primers are present at about 0.002 μM to about 1.0 μM and the target amplification primers are present at about 0.1 μM to about 2.0 μM.

### Embodiment 7

The method of any one of the previous embodiments, wherein the sequence-specific probes are present at about 0.01 μM to about 1.0 μM in the second reaction mix.

### Embodiment 8

The method of any one of the previous embodiments, wherein all target enrichment primers are present in the first reaction mix at substantially the same concentration.

### Embodiment 9

The method of any one of the previous embodiments, wherein all target amplification primers are present in the second reaction mix at substantially the same concentration.

### Embodiment 10

The method of any one of the previous embodiments, wherein the step a) amplification reaction includes at least two complete cycles of a target enrichment process and the step b) amplification reaction includes at least two complete cycles of a target amplification process.

### Embodiment 11

The method of embodiment 10, wherein the target enrichment process comprises two stages wherein the first stage comprises about 10 seconds to about 1 minute at about 92° C. to about 95° C., about 30 seconds to about 2.5 minutes at about 50° C. to about 60° C., and about 30 seconds to about 1.5 minutes at about 70° C. to about 75° C.; and wherein the second stage comprises about 10 seconds to about 1 minute at about 92° C. to about 95° C., and about 30 seconds to about 2.5 minutes at about 68° C. to about 75° C.

### Embodiment 12

The method of embodiment 11, wherein the step a) amplification reaction includes reverse transcription.

### Embodiment 13

The method of embodiment 12, wherein reverse transcription comprises about 2 to about 20 minutes at about 45° C. to about 55° C.

### Embodiment 14

The method of embodiment 10, wherein the target amplification process comprises about 15 seconds to about 1 minute at about 92° C. to about 95° C., and about 30 seconds to about 1.5 minutes at about 55° C. to about 65° C.

### Embodiment 15

The method of embodiment 1, wherein the step a) amplification reaction includes at least 15 complete cycles of a target enrichment process and at least 5 complete cycles of a selective amplification process, and wherein the step b) amplification reaction includes at least 15 complete cycles of a target amplification process.

### Embodiment 16

The method of embodiment 11, wherein the length of each of the first target enrichment primers is about 10 to about 100 nucleotides and the length of each of the second target enrichment primers is about 10 to about 100 nucleotides.

### Embodiment 17

The method of any one of the previous embodiments, wherein the first reaction mix comprises at least 10 distinct pairs of target enrichment primers.

### Embodiment 18

The method of any one of the previous embodiments, wherein the second reaction mix comprises two or more pairs of target amplification primers.

### Embodiment 19

The method of any one of the previous embodiments, wherein at least one primer set hybridizes to a viral or bacterial nucleotide sequence or a genetic determinant of antibiotic resistance.

### Embodiment 20

The method of embodiment 19, wherein the bacteria are selected from the group consisting of Helicobacter species, Neisseria meningitides, Haemophilus influenzae, Escherichia coli, Listeria monocytogenes, Mycoplasma pneumoniae, Streptococcus pneumoniae, and Streptococcus agalactiae and the viruses are selected from the group consisting of enteroviruses, coxsackievirus A, coxsackievirus B, parechovirus, and West Nile virus.

### Embodiment 21

The method of any one of the previous embodiments, wherein the genetic determinant of antibiotic resistance is clarithromycin resistance.

### Embodiment 22

The method of any one of the previous embodiments, wherein the thermostable DNA polymerase is Thermophilus aquaticus DNA polymerase.

### Embodiment 23

The method of any one of the previous embodiments, wherein the second reaction mix comprises sequence specific probes directed to at least 3 different target sequences.

### Embodiment 24

The method of embodiment 23, wherein the second reaction mix comprises sequence specific probes directed to at least 10 different target sequences.

### Embodiment 25

The method of embodiment 24, wherein the second reaction mix comprises sequence specific probes directed to at least 20 different target sequences.

### Embodiment 26

The method of embodiment 25, wherein the second reaction mix comprises sequence specific probes directed to at least 30 different target sequences.

### Embodiment 27

The method of any one of the previous embodiments, wherein the first amplification products from step a) are diluted at least five fold relative to the concentration of first amplification products at the end of step a).

### Embodiment 28

The method of embodiment 27, wherein the first amplification products from step a) are diluted at least ten fold relative to the concentration of first amplification products at the end of step a).

### Embodiment 29

The method of any one of the previous embodiments, wherein the quantitative amplification is a real-time amplification.

### Embodiment 30

A method for quantitative multiplex amplification, the method comprising:
a. amplifying a plurality of target sequences in a first reaction mix, wherein for each target sequence in the plurality, the first reaction mix comprises:
   i. a first pair of target enrichment primers comprising a forward outer (Fₒ) and a reverse outer (Rₒ) primer that each hybridize to a sequence adjacent to the target sequence; and
   ii. a second pair of target enrichment primers comprising a forward inner (Fᵢ) and a reverse inner (Rᵢ) primer, each of which hybridize to a portion of the target sequence, wherein Fᵢ primers comprise a tag comprising a sequence complementary to a portion of one of a pair of target amplification primers and wherein Rᵢ primers comprise a tag comprising a sequence complementary to a portion of the other of the pair of target amplification primers; and
wherein the step a) amplification generates a plurality of first amplification products; and
b. amplifying the plurality of target sequences in a cycler containing a second reaction mix, wherein the second reaction mix comprises:
   i. the target amplification primers, in which the pair of target amplification primers comprises a forward super primer (FSP) and a reverse super primer (RSP), each of which binds to its corresponding tag on the first amplification products;
   ii. a dilution of the first amplification products from step a), diluted at least two fold relative to the concentration of first amplification products at the end of step a); and
   iii. a fluorescent dye that emits a more intense fluorescence at a given wavelength when bound to double-stranded DNA (dsDNA) than when not bound to dsDNA,
wherein the cycler is equipped with filters responsive to a plurality of fluorescence wavelengths, and wherein an illumination source periodically illuminates the second reaction mix for detection.

### Embodiment 31

The method of embodiment 30, wherein FSP binds the complement of the tag on Fᵢ and RSP binds the complement of the tag on Rᵢ.

### Embodiment 32

The method of any one of the previous embodiments, wherein the length of each of the first pair of target enrichment primers is about 10 to about 100 nucleotides.

### Embodiment 33

The method of any one of the previous embodiments, wherein the length of each of the second pair of target enrichment primers is about 10 to about 100 nucleotides.

### Embodiment 34

The method of any one of the previous embodiments, wherein the length of each of the target amplification primers is about 10 to about 100 nucleotides.

### Embodiment 35

The method of any one of the previous embodiments, wherein the target enrichment primers are present at about 0.002 μM to about 1.0 μM and the target amplification primers are present at about 0.1 μM to about 2.0 μM.

### Embodiment 36

The method of embodiment 35, wherein all target enrichment primers are present in the first reaction mix at substantially the same concentration.

### Embodiment 37

The method of embodiment 35, wherein all target amplification primers are present in the second reaction mix at substantially the same concentration.

### Embodiment 38

The method of embodiment 30, wherein the step a) amplification reaction includes at least 15 complete cycles of a target enrichment process and the step b) amplification reaction includes at least 15 complete cycles of a target amplification process.

### Embodiment 39

The method of embodiment 38, wherein the target enrichment process comprises two stages wherein the first stage comprises about 10 seconds to about 1 minute at about 92° C. to about 95° C., about 30 seconds to about 2.5 minutes at about 50° C. to about 60° C., and about 30 seconds to about 1.5 minutes at about 70° C. to about 75° C.; and wherein the second stage comprises about 10 seconds to about 1 minute at about 92° C. to about 95° C., and about 30 seconds to about 2.5 minutes at about 68° C. to about 75° C.

### Embodiment 40

The method of embodiment 39, wherein the step a) amplification reaction includes reverse transcription.

### Embodiment 41

The method of embodiment 40, wherein reverse transcription comprises about 2 to about 20 minutes at about 45° C. to about 55° C.

### Embodiment 42

The method of embodiment 38, wherein the target amplification process comprises about 15 seconds to about 1 minute at about 92° C. to about 95° C., and about 30 seconds to about 1.5 minutes at about 55° C. to about 65° C.

### Embodiment 43

The method of any one of the previous embodiments, wherein the step a) amplification reaction includes at least two complete cycles of a target enrichment process and at least two complete cycles of a selective amplification process, and wherein the step b) amplification reaction includes at least two complete cycles of a target amplification process.

### Embodiment 44

The method of embodiment 43, wherein the length of each of the first target enrichment primers is about 10 to about 100 nucleotides and the length of each of the second target enrichment primers is about 10 to about 100 nucleotides.

### Embodiment 45

The method of any one of the previous embodiments, wherein the first reaction mix comprises at least 10 distinct pairs of target enrichment primers.

### Embodiment 46

The method of any one of the previous embodiments, wherein the second reaction mix comprises two or more pairs of target amplification primers.

### Embodiment 47

The method of any one of the previous embodiments, wherein at least one primer set hybridizes to a viral or bacterial nucleotide sequence or a genetic determinant of antibiotic resistance.

### Embodiment 48

The method of embodiment 47, wherein the bacteria are selected from the group consisting of Helicobacter species, Neisseria meningitides, Haemophilus influenzae, Escherichia coli, Listeria monocytogenes, Mycoplasma pneumoniae, Streptococcus pneumoniae, and Streptococcus agalactiae and the viruses are selected from the group consisting of enteroviruses, coxsackievirus A, coxsackievirus B, parechovirus, and West Nile virus.

### Embodiment 49

The method of embodiment 47, wherein the genetic determinant of antibiotic resistance is clarithromycin resistance.

### Embodiment 50

The method of any one of the previous embodiments, wherein the thermostable DNA polymerase is Thermophilus aquaticus DNA polymerase.

### Embodiment 51

The method of any one of the previous embodiments, wherein the first amplification products from step a) are diluted at least five fold relative to the concentration of first amplification products at the end of step a).

### Embodiment 52

The method of embodiment 51, wherein the first amplification products from step a) are diluted at least ten fold relative to the concentration of first amplification products at the end of step a).

### Embodiment 53

The method of any one of the previous embodiments, further comprising c) gradually melting the amplicons by raising temperature in the cycler no faster than about 0.5 C° per second.

### Embodiment 54

The method of embodiment 53, comprising measuring fluorescence at each 0.5 C° increase.

### Embodiment 55

The method of embodiment 30, wherein:
(a) the fluorescent dye is N′,N′-dimethyl-N-[4-[(E)-(3-methyl-1,3-benzothiazol-2-ylidene)methyl]-1-phenylquinolin-1-ium-2-yl]-N-propylpropane-1,3-diamine and the given wavelength is 520 nm; or
(b) the fluorescent dye is AOAO-12 and the given wavelength is 530 nm; or
(c) the fluorescent dye is ATTO-633 and the given wavelength is 657 nm; or
(d) the fluorescent dye is ATTO-647N and the given wavelength is 669 nm; or
(e) the fluorescent dye is ATTO-655 and the given wavelength is 684 nm.

### EXAMPLES

### Example 1. Validation of Probes

Nucleotide probes were designed to target Helicobacter pylori 23S gene, Helicobacter pylori ureA gene, and an internal control based on an Arabidopsis thaliana sequence. In particular, three point mutation probes were designed: one specific to wild-type 23S; one specific to an A2142G point mutant; and one specific to an A2143G point mutant. The wild-type probe was labeled with VIC® dye at one end and a QSY® quencher at the other end. The A2142G probe was labeled with NED® dye at one end and MGB quencher at the other end. The A2143G probed was labeled with FAM® at one end and MGB quencher at the other end. The ureA probe was labeled with JUN dye at one end and QSY quencher at the other end. The A. thaliana probe was labeled with CY5 dye at one end and Iowa Black quencher at the other end. Each of these probes was tested in amplification using primers specific to the 23S gene, the ureA gene, and the apx1 gene of A. thalina, with template DNA from wild-type H. pylori (CCUG 38770 and CCUG 38771), an H. pylori strain bearing a A2143G point mutation (CCUG 38772), a synthetic plasmid bearing the A2142G sequence (A2142G pDNA), and a synthetic plasmid bearing a select region of A. thaliana apx1. No detection of any targets, determined by absence of a determined crossing threshold cycle (Ct), was observed with the no template control (NTC) indicating no contamination. For templates CCUG 38770, CCUG 38771, and CCUG 38772 containing the ureA gene, amplification was observed with the ureA probe indicating detection of the ureA gene. Amplification was observed by noting a determined Ct. No detection was made for ureA with A2142G pDNA as this template does not contain the ureA gene. For templates CCUG 38770 and CCUG 38771 containing wild type 23S gene, the cycle where the fluorescence of the 23S wild type (WT) probe crosses the threshold (Ct) was at least 6.5 Cts lower than the mutant A2142G and A2143G probes. This Ct shift between wild type and mutant probes is interpreted as being wild type as wild type template with WT probe is expected to cross the threshold at a lower cycle number, thus have a lower Ct. Template CCUG 38772 with an A2143G 23S gene mutation was observed to only have a detectable Ct value with the A2143G probe with regards to the A2142G probe and the WT probe. The A2142G template with an A2142G 23S gene mutation was observed to have a lower Ct value for the A2142G probe with regards to the A2143G probe and is interpreted to be an A2142G detection by the same logic of the Ct shift. Internal control template for apx1 was added to all reactions except the NTC and is observed with Ct values for the APX1 Internal Control Probe demonstrating lack of PCR inhibition. Results from this test are shown in Table 1.

TABLE 1

| | Avg. Ct | | | | |
|---|---|---|---|---|---|
| | | | APX1 | | |
| | A2142G | A2143G | Internal | ureA | WT |
| Template | Probe | Probe | Control | Probe | probe |
| No Template Control | — | — | — | — | — |
| H. pylori CCUG 38770 | 31.5 | 19.3 | 19.4 | 10.1 | 12.0 |
| 0.1 ng/μL WT | | | | | |
| H. pylori CCUG 38770 | 36.0 | 22.5 | 19.2 | 13.7 | 15.8 |
| 0.01 ng/μL WT | | | | | |
| H. pylori CCUG 38771 | 30.8 | 20.1 | 19.4 | 11.2 | 13.1 |
| 0.1 ng/μL WT | | | | | |
| H. pylori CCUG 38771 | 32.8 | 22.6 | 19.1 | 14.1 | 16.1 |
| 0.01 ng/μL WT | | | | | |
| H. pylori CCUG 38772 | — | 13.0 | 19.2 | 18.4 | — |
| 0.1 ng/μL A2143G | | | | | |
| H. pylori CCUG 38772 | — | 16.3 | 19.3 | 21.4 | — |
| 0.01 ng/μL A2143G | | | | | |
| A2142G plasmid | 21.2 | 27.0 | 19.7 | — | — |
| 100000 copies/μL | | | | | |

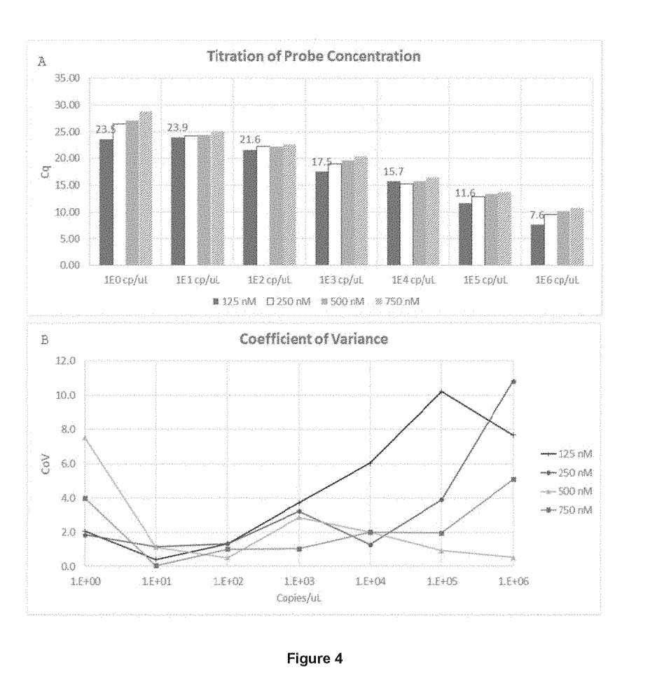

The WT probe was tested at 125 nM, 250 nM, 500 nM, & 750 nM to determine the optimum concentration. The results of these tests are shown in FIGS. 4A & B. Based on these results, further experiments were conducted at a probe concentration of 250 nM.

### Example 2. Real-Time Quantification with Multiplex PCR

As a preliminary to assembling PCR reactions, laminar flow hoods were wiped down with DNA, RNA, DNase, and RNase surface decontaminants, and a 10% bleach solution. The same hoods were then decontaminated by a 15 minute ultraviolet light exposure. All experimental procedures were carried out in these cleaned and decontaminated hoods.

H. pylori template DNA was extracted from H. pylori CCUG 38770, wild type for 23S, H. pylori CCUG 38771, wild type for 23S, and H. pylori CCUG 38772, and an A2143G mutant strain. A synthetic plasmid with a select segment of the H. pylori 23S gene designed with the A2142G mutation was transformed into E. coli. Subsequent processing yielded a quantity of the synthetic plasmid referred to as A2142G pDNA. The template DNA diluted to 0.1 ng/μL or 0.01 ng/μL. A2142G pDNA was diluted to 1×10⁴ copies/μL. Fₒ, Fᵢ, Rₒ, and Rᵢ primers specific for the H. pylori 23S gene and the H. pylori ureA gene were mixed and diluted to achieve a final concentration of about 0.16 pmol/μL.

Enzyme mix, 2.4 μL of primer mix, 4 μL of template DNA, and nuclease-free water were added to each reaction tube containing to a total volume of 20 μL per reaction, and the tubes were thermally cycled in an Applied Biosystems GeneAmp 9700 thermocycler under the following conditions: (activation) 95° C. for 2 min; (enrichment) 15 cycles of 94° C. for 30 sec, 55° C. for 2 min, 72° C. for 1 min; and (tagging) 6 cycles of 94° C. for 30 sec, 72° C. for 1.5 min.

After the tagging, the reactions were removed from the thermocycler. Inside a hood, 180 μL of nuclease-free water were added to each reaction to achieve a ten-fold dilution.

A probe, primer, and enzyme mix was then assembled containing: a probe directed to wild-type 23S (labeled with VIC); a probe directed to an A2142G mutant 23S allele (labeled with NED); a probe directed to an A2143G mutant 23S allele (labeled with FAM); a probe directed to ureA (labeled with JUN); forward and reverse super primers; and enzyme THERMO FISHER TaqMan master mix. The probes were diluted to achieve a final concentration of 250 nM and the primers to a final concentration of 900 nM. Six microliters of this probe/primer/enzyme mix was mixed with 4 μL of the tagged-target dilution, and the resulting reaction mix was thermally cycled in the thermocycler under the following conditions: 50° C. for 2 min; 95° C. for 2 min; 40 cycles of 95° C. for 15 sec, 62° C. for 1 min.

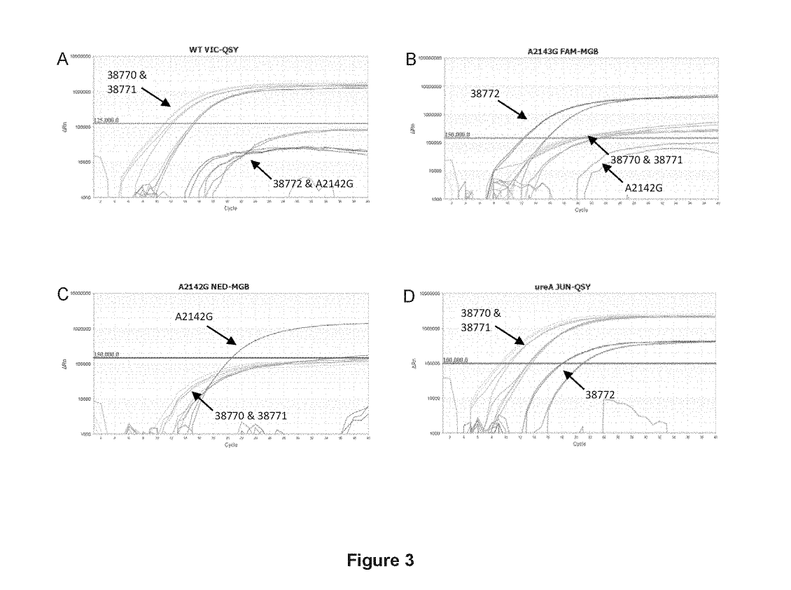

FIG. 3 panels A-D show the results of this trial. As can be seen, each strain amplified and was detectable in real time. The specificity of the 23S probes were demonstrated by the Ct shift observed for wild-type versus mutant strains.

The above examples are merely illustrative, and do not limit this disclosure in any way. All patents and patent applications cited herein are incorporated by reference to the extent allowed. The references discussed herein are provided solely for their disclosure prior to the filing date of the present application. Nothing herein is to be construed as an admission that the inventors are not entitled to antedate such disclosure by virtue of prior disclosure.

What is claimed is:

1. A method for quantitative multiplex amplification, the method comprising:
a) amplifying a plurality of target sequences in a first reaction mix, wherein for each target sequence in the plurality, the first reaction mix comprises:
i) a first pair of target enrichment primers comprising a forward outer (Fₒ) and a reverse outer (Rₒ) primer that each hybridize to a sequence adjacent to the target sequence; and
ii) a second pair of target enrichment primers comprising a forward inner (Fᵢ) and a reverse inner (Rᵢ) primer, each of which hybridize to a portion of the target sequence, wherein Fᵢ primers comprise a tag comprising a sequence complementary to a portion of one of a pair of target amplification primers and wherein Rᵢ primers comprise a tag comprising a sequence complementary to a portion of the other of the pair of target amplification primers; and
wherein the step a) amplification generates a plurality of first amplification products; and
b) amplifying the plurality of target sequences in a cycler containing a second reaction mix, wherein the second reaction mix comprises:
i) the target amplification primers, in which the pair of target amplification primers comprises a forward super primer (FSP) and a reverse super primer (RSP), each of which binds to its corresponding tag on the first amplification products;
ii) a dilution of the first amplification products from step a), diluted at least two fold relative to the concentration of first amplification products at the end of step a);
iii) a thermostable DNA polymerase with 5′ to 3′ exonuclease activity; and
iv) a plurality of sequence-specific probes, wherein there is at least one probe complementary to each target sequence in the plurality, and wherein each sequence-specific probe comprises at least one fluorophore and a quencher, wherein the at least one fluorophore responds to an excitation wavelength by emitting a first fluorescence, and wherein the quencher quenches the first fluorescence prior to hydrolysis of the probe,
wherein the cycler is equipped with filters responsive to a plurality of first fluorescence wavelengths, and wherein an illumination source periodically illuminates the second reaction mix for detection.

2. The method of claim 1, wherein FSP binds the complement of the tag on Fᵢ and RSP binds the complement of the tag on Rᵢ.

3. The method of claim 1, wherein the first and second pairs of target enrichment primers are each about 10 to about 100 nucleotides in length.

4. The method of claim 1, wherein the target enrichment primers are present at about 0.002 μM to about 1.0 μM and the target amplification primers are present at about 0.1 μM to about 2.0 μM.

5. The method of claim 1, wherein the sequence-specific probes are present at about 0.01 μM to about 1.0 μM in the second reaction mix.

6. The method of claim 4, wherein all target enrichment primers are present in the first and second reaction mixes at substantially the same concentration.

7. The method of claim 1, wherein the step a) amplification reaction includes at least two complete cycles of a target enrichment process and the step b) amplification reaction includes at least two complete cycles of a target amplification process.

8. The method of claim 7, wherein the step a) amplification reaction includes reverse transcription.

9. The method of claim 1, wherein the step a) amplification reaction includes at least 15 complete cycles of a target enrichment process and at least 5 complete cycles of a selective amplification process, and wherein the step b) amplification reaction includes at least 15 complete cycles of a target amplification process.

10. The method of claim 1, wherein the first reaction mix comprises at least 10 distinct pairs of target enrichment primers.

11. The method of claim 1, wherein the second reaction mix comprises two or more pairs of target amplification primers.

12. The method of claim 1, wherein at least one primer set hybridizes to a viral or bacterial nucleotide sequence or a genetic determinant of antibiotic resistance.

13. The method of claim 13, wherein the bacteria are selected from the group consisting of Helicobacter species, Neisseria meningitides, Haemophilus influenzae, Escherichia coli, Listeria monocytogenes, Mycoplasma pneumoniae, Streptococcus pneumoniae, and Streptococcus agalactiae and the viruses are selected from the group consisting of enteroviruses, coxsackievirus A, coxsackievirus B, parechovirus, and West Nile virus.

14. The method of claim 13, wherein the genetic determinant of antibiotic resistance is clarithromycin resistance.

15. The method of claim 1, wherein the thermostable DNA polymerase is Thermophilus aquaticus DNA polymerase.

16. The method of claim 1, wherein the second reaction mix comprises sequence specific probes directed to at least 3 different target sequences.

17. The method of claim 17, wherein the second reaction mix comprises sequence specific probes directed to at least 10 different target sequences.

18. The method of claim 1, wherein the first amplification products from step a) are diluted at least five fold relative to the concentration of first amplification products at the end of step a).

19. The method of claim 18, wherein the first amplification products from step a) are diluted at least ten fold relative to the concentration of first amplification products at the end of step a).

20. A method for quantitative multiplex amplification, the method comprising:
a) amplifying a plurality of target sequences in a first reaction mix, wherein for each target sequence in the plurality, the first reaction mix comprises:
i) a first pair of target enrichment primers comprising a forward outer (Fₒ) and a reverse outer (Rₒ) primer that each hybridize to a sequence adjacent to the target sequence; and
ii) a second pair of target enrichment primers comprising a forward inner (Fᵢ) and a reverse inner (Rᵢ) primer, each of which hybridize to a portion of the target sequence, wherein Fᵢ primers comprise a tag comprising a sequence complementary to a portion of one of a pair of target amplification primers and wherein Rᵢ primers comprise a tag comprising a sequence complementary to a portion of the other of the pair of target amplification primers; and
wherein the step a) amplification generates a plurality of first amplification products; and
b) amplifying the plurality of target sequences in a cycler containing a second reaction mix, wherein the second reaction mix comprises:
i) the target amplification primers, in which the pair of target amplification primers comprises a forward super primer (FSP) and a reverse super primer (RSP), each of which binds to its corresponding tag on the first amplification products;
ii) a dilution of the first amplification products from step a), diluted at least two fold relative to the concentration of first amplification products at the end of step a); and
iii) a fluorescent dye that emits a more intense fluorescence at a given wavelength when bound to double-stranded DNA (dsDNA) than when not bound to dsDNA,
wherein the cycler is equipped with filters responsive to a plurality of first fluorescence wavelengths, and wherein an illumination source periodically illuminates the second reaction mix for detection.
